(19) United States
(12) Patent Application Publication
(10) Pub. No.: US 2016/0031958 A1
(43) Pub. Date: Feb. 4, 2016

(54) METHODS AND COMPOSITIONS USEFUL IN MANIPULATING THE STABILITY OF RE1 SILENCING TRANSCRIPTION FACTOR

(71) Applicant: Edmund Nesti, Beltsville, MD (US)

(72) Inventor: Edmund Nesti, Beltsville, MD (US)

(73) Assignee: OREGON HEALTH & SCIENCE UNIVERSITY, Portland, OR (US)

(21) Appl. No.: 14/815,598

(22) Filed: Jul. 31, 2015

Related U.S. Application Data
(60) Provisional application No. 62/032,438, filed on Aug. 1, 2014.

(51) Int. Cl. C07K 14/47; C07K 16/18; G01N 33/68

(52) U.S. Cl. CPC C07K 14/4703; G01N 33/6893; C07K 16/18; G01N 2333/4704

(57) ABSTRACT

Disclosed are methods of screening for compounds that promote REST degradation by inhibiting the activity of the CDTSP1 phosphorylase including fluorescent and antibody based screens. Also disclosed are peptides that promote REST stabilization as well as antibodies that recognize REST phosphorylated at serine 861 and serine 864.

### FIELD

Generally, the field is manipulating the stability of transcription factors. More specifically, the field is screening of compounds that manipulate the stability of the RE1 silencing transcription factor.

### BACKGROUND

The RE1 Silencing Transcription factor (REST) is a transcriptional repressor that suppresses neuronal gene expression in non-neural cells, such as fibroblasts, as well as in neural progenitors (Chong J A et al, Cell 80, 949-957 (1995); Schoenherr C J and Anderson D J, Science 267, 1360-1363 (1995); and Ballas N et al, Cell 121, 645-657 (2005); all of which are incorporated by reference herein). Its targets represent genes required for the terminally differentiated neuronal cell phenotype, including genes encoding voltage and ligand dependent ion channels, their receptors, growth factors, and axonal-guidance proteins (Bruce A W et al, Proc Natl Acad Sci USA 101, 10458-10463 (2004); Conaco C et al, Proc Natl Acad Sci USA 103, 2422-2427 (2006); Mortazavi A et al, Genome Res 16, 1208-1221 (2006); and Otto S J et al, J Neurosci 27, 6729-6739 (2007); all of which are incorporated by reference herein). Thus, during neurogenesis, REST is progressively down regulated to allow elaboration of the mature neuronal phenotype (Ballas et al, 2005 supra). The importance of this event is demonstrated by gain-of-function studies that indicate the persistence of REST impedes terminal neuronal differentiation (Mandel G et al, Proc Natl Acad Sci USA 108, 16789-16794 (2011) and Gao Z et al, J Neurosci 31, 9772-9786 (2011); both of which are incorporated by reference herein). Nonetheless, precisely how REST itself is regulated still remains an open question. Relatively little is known either about its transcriptional or post-transcriptional regulation (Ballas N et al 2005 supra; Ballas N et al, Neuron 31, 353-365 (2001); and Kojima T et al, Brain Res Mol Brain Res 90, 174-186 (2001); all of which are incorporated by reference herein). In contrast, several studies have focused on post-translational regulation of REST, but the identity of the signaling molecules involved has received little attention.

In neural progenitors and Human Embryonic Kidney (HEK) cells, rapid REST turnover is mediated by targeting to a proteasomal pathway (Ballas et al, 2005 supra; Guardavacarro D et al, Nature 452, 365-369 (2008); and Westbrook T F et al, Nature 452, 370-374 (2008); all of which are incorporated by reference herein). REST degradation during neuronal differentiation in culture requires interaction with βTrCP, an E3-ubiquitin ligase, for targeting to the proteasome (Westbrook et al, 2008 supra). βTrCP was also required for cell cycle-dependent degradation of REST in HEK cells (Guardavaccaro et al, 2008 supra). Two adjacent phosphorylated peptides in the C-terminal domain of REST were identified as βTrCP substrates in these studies, and function as degrons. The kinases responsible for the phosphorylation and driving degron activity were not identified.

### SUMMARY

Disclosed herein is a new proline-directed phosphorylation site, ⁸⁶¹SPP⁸⁶⁴SP (SEQ ID NO: 1), in a domain of REST that regulates REST stability. This site lies N-terminal to the degrons identified previously.

Disclosed herein is that phosphorylation of serines at 861/864 regulates βTrCP binding to REST. Using a reporter peptide comprising S861/S864, as well as full length REST protein, phosphorylation in response to perturbations of the ERK pathway is monitored and binding and activity of the proline isomerase Pin1 and the RNA polymerase C-terminal domain phosphatase CTDSP1 are demonstrated.

Disclosed herein is the role of REST S861/864 sites in destabilizing REST and promoting terminal neuronal differentiation of primary cultures of cortical neural progenitors.

Disclosed herein are methods of selecting test compounds that promote REST degradation. One such method involves adding a test compound, a peptide comprising SEQ ID NO: 1 and an active CTDSP1 protein to a mixture. Both serines of SEQ ID NO: 1 are phosphorylated on the peptide. In this method, a test compound that results in the maintenance of phosphorylation of the serines of SEQ ID NO: 1 is a compound that promotes REST degradation. In further examples of this method, the peptide is at least 50% identical to SEQ ID NO: 2. In still further examples, this method further comprises adding a composition that fluoresces in the presence of free phosphate to the mixture. In this method, a test compound that results in less fluorescence relative to a negative control is a compound that promotes REST degradation.

Another method of selecting a test compound that promotes REST degradation involves adding a test compound; a peptide comprising SEQ ID NO: 1; an antibody that specifically binds phosphorylated SEQ ID NO: 1 but does not bind unphosphorylated SEQ ID NO: 1; and an active CTDSP1 protein to a mixture. The method further comprises detecting binding of the antibody to the peptide. A test compound that results in less binding of the antibody to the peptide relative to a negative control is a compound that promotes REST degradation. The antibody can be a monoclonal or a polyclonal antibody. Antibody binding can be determined using any method including methods involving surface plasmon resonance.

Also disclosed are methods of stabilizing REST in a cell. The methods involve contacting the cell with a peptide comprising SEQ ID NO: 1, thereby stabilizing REST in the cell. The cell could comprise a plasmid that results in the expression of the peptide. Additionally, the cell with stabilized REST could be in vivo.

Also disclosed are compositions such as a peptide of SEQ ID NO: 2 further comprising a protein tag. The protein tag can be a FLAG® tag. In some examples, the peptide is phosphorylated at the serines of SEQ ID NO: 1.

Also disclosed are monoclonal and polyclonal antibodies that specifically bind the peptide of SEQ ID NO: 2 when the serines of SEQ ID NO: 1 are phosphorylated, but not when the serines of SEQ ID NO: 1 are not phosphorylated, including antibodies produced through immunization with SEQ ID NO: 4.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE SEVERAL VIEWS OF THE DRAWINGS

Some of the drawings herein are better understood when presented in color, which is not available in patent application publications. However, Applicants consider the color drawings to be part of the original disclosure and reserve the right to present color versions of the drawings herein in later proceedings.

FIG. 1A, FIGS. 1B and 1C collectively show that mutating REST Serines 861/864 modulates REST protein stability without affecting chromatin binding.

FIG. 1A is a schematic of the predicted primary structure of human REST protein showing the disclosed phosphorylated site (861/864) relative to the previously identified degrons at 1009/1013 and 1024/1027/1030.

FIG. 1B is a plot showing that REST degradation in HEK293T cells is delayed in S861A/S854A phosphorylation mutants. CHX on the x-axis indicates the number of hours incubated in 25 mM cyclohexamide. The differences between unmutated (WT) REST and mutant S861/864A (interchangeably referred to herein as S861A/S864A) REST are significant (SEM; p<0.05) at every time point measured. The differences between WT and mutant 51024/1027/1030A (interchangeably referred to herein as S1024A/S1027A/S1030A) are also significant (SEM; p<0.05) at 1.5 and 3 h. There is no significant difference between the two mutants at any time point (Kruskal-Wallis test). The degradation data were fit with monoexponential functions yₒ+A (−inv Tau−x) with y-intercept set to 100%.

FIG. 1C is a histogram showing no change in occupancy of WT and S861A/S864A mutant FLAG-REST at the RE1 site of the transcription factor NPAS4 gene in HEK293T cells. The Four and One Half Lim Domains 5 gene (FHL5) lacks an RE1 site.

FIG. 2A, FIG. 2B, FIG. 2C and FIG. 2D collectively show that mutating REST serines 861 and 864 to alanines disrupts bTrCP-binding to the downstream degrons.

FIG. 2A is an image of a Western blot analysis showing reduced levels of mutant REST compared to unmutated REST in GST-βTrCP complexes. HA-fusion cDNAs were co-transfected into HEK293T cells along with GST-βTrCP and FLAG-Dominant Negative-Cull to stabilize REST. GST-βTrCP complexes were isolated from cell extracts on glutathione agarose, and probed with anti-GST and anti-HA antibodies.

FIG. 2B is a histogram summarizing the results of FIG. 2A. Band intensities of HA-WT and mutant REST proteins were determined by fluorescence intensity and normalized to GST-βTrCP levels in the glutathione IP. (n=10 for each sample, ***p=0.0002, Holm-Sidak's multiple comparisons test, error bars represent 95% confidence intervals.)

FIG. 2C is an image of a Western blot resulting from co-immunoprecipitation analysis showing that the REST degron mutant E1009A/S1013A/S1024A/S1027A/S1030A (E1009A/S1013/24/27/30A), which is unmutated at S861/S864, does not bind to GST-βTrCP. HA-REST cDNAs were transfected into HEK293T cells along with GST-βTrCP, and FLAG-Dominant Negative Cull to stabilize REST. Cell extracts were collected on glutathione agarose and probed with anti-HA or anti-GST antibodies.

FIG. 2D is an image of a Western blot showing increased CoREST in complexes with REST mutant (unmutated vs. S861A/S864A, n=4, p=0.0027, ratio paired t test).

FIG. 3A and FIG. 3B collectively show that a REST-GFP fusion peptide (amino acids 810-910) detects phosphorylation on serines 861 and 864.

FIG. 3A is a schematic showing the location of REST (810-910) and control (595-694) peptides within the whole REST protein. Potentially phosphorylated residues are in bold.

FIG. 3B is an image of a native gel showing that the REST 810-910 peptide is phosphorylated primarily on serines 861/864 in HEK293T cells. Extracts were prepared from cells transfected with cDNAs for REST (810-910), mutated REST (810-910), or control (595-694) peptides were resolved on native gels and analyzed by direct GFP fluorescence.

FIG. 4A, FIG. 4B, FIG. 4C, and FIG. 4D collectively show that peptidyl-prolyl cis/trans isomerase (Pint) activity at serines 861/864 promotes REST degradation.

FIG. 4A is an image of a Western blot of co-immunoprecipitation analysis showing Pin1 is present in REST (810-910) immunoprecipitates in a phospho-dependent manner. HEK293T cells were transfected with Pin1 cDNA and either WT or mutant (S861A/S864A) FLAG-REST (810-910) or FLAG-control (595-694) peptide cDNAs (FIG. 3). After 48 hours, the cells were extracted and immunoprecipitated. Western blots were probed with the indicated antibodies.

FIG. 4B is an image of a Western blot of a co-immunoprecipitation analysis showing REST forms complexes with Pint. HEK293T cells were transfected with REST and FLAG-Pin1 cDNAs for 48 h then treated with MG132 for 4 h prior to extraction and immunoprecipitation. Western blots were probed with the indicated antibodies.

FIG. 4C is an image of a Western blot showing higher REST protein levels in the presence of the Pin1 inhibitor, PiB. PC12 tet-on FLAG-REST cells were induced 24 h with doxycycline, treated with PiB or vehicle for 7.5 h, and then extracted for analysis. Blots were probed with anti-FLAG (REST) and α-tubulin (loading control).

FIG. 4D is an image of a Western blot of a coimmunoprecipitation analysis showing inhibition of Pin1 results in diminished βTrCP binding to REST comparable to S861A/S864A. HEK293T cells were transfected with FLAG-REST (unmutated or S861A/S864A) and GST-βTrCP for 48 h prior to treatment with PiB for 7.5 h and MG132 for 4 h, and then immunoprecipitated. Western blots were probed with anti-FLAG or anti-GST.

FIG. 5A, FIG. 5B, FIG. 5C, FIG. 5D, and FIG. 5E collectively show that EGF, RAS and ERK signaling phosphorylates Serines 861/864 on REST.

FIG. 5A is an image of a native gel showing that EGF induces phosphorylation of S864 in REST that is inhibited by treatment with PD184352, a MEK inhibitor. To interrogate this serine residue, HEK293T cells were transfected with the REST (810-910)-GFP peptide with alanine mutations at S856, S861, and/or S864 as indicated. (S856/864A is S856A/S864A with S861 unmutated; S856/861A is S856A/S861A with S864 unmutated; S861/864A is S861A/S864A with S856 unmutated). Cells were treated or not with PD184352 for 20 min, followed by treatment with recombinant EGF or vehicle for further 10 min prior to analysis.

FIG. 5B is an image of a native gel showing that REST S861/S864 is phosphorylated by H-Ras. HEK293T cells were co-transfected with active H-Ras and indicated REST (810-910) constructs.

FIG. 5C is an image of a native gel showing partial loss of S861/S864 phosphorylation in WT REST peptide after treating transfected HEK293T cells with 10 mM PD184352 for 4 hours.

FIG. 5D is an image of a Western blot showing increased FLAG-REST protein levels after treatment with PD184352 or vehicle for 30 min.

FIG. 5E is an image of an In vitro kinase assay showing direct phosphorylation of REST by recombinant ERK2. The indicated GST-REST peptides were expressed and purified from bacteria then incubated with either recombinant ERK2 protein or HEK293T whole cell lysate. The blot was probed with GST antibody and REST phospho-S861/864 polyclonal antibody.

FIG. 6A, FIG. 6B, FIG. 6C, and FIG. 6D collectively show that CTDSP1 dephosphorylates REST at Serines 861/864 and stabilizes REST

FIG. 6A is an image of a Western blot analysis showing that REST forms immunocomplexes with CTDSP1. HEK293T cells were transfected with either WT or mutant HA-REST cDNAs together with FLAG-CTDSP1, and treated with MG132 for 4 h prior to FLAG IP. Western blots were probed for REST and FLAG.

FIG. 6B is an image of a Western blot analysis showing loss of S861/S864 phosphorylation in full length REST after cotransfection of FLAG-CTDSP1. HEK293T cells were transfected with FLAG-REST and FLAG-CTDSP1 or empty vector and treated with MG132 for 4 hours to stabilize REST. The blot was probed with FLAG antibody and polyclonal REST phospho-S861/S864 antibody.

FIG. 6C is an image of a native gel showing partial loss of S861/S864 phosphorylation on REST-GFP peptide after co-transfection with CTDSP1 cDNA into HEK293T cells. Peptides were visualized by direct GFP fluorescence.

FIG. 6D is an image of a Western blot analysis showing stabilized endogenous REST protein after transfection of CTDSP1 cDNA into HEK293T cells. α-tubulin is the loading control.

FIG. 7A, FIG. 7B, and FIG. 7C collectively show that the expression of REST (810-910) peptide in neurospheres stabilizes endogenous REST protein.

FIG. 7A is a photomicrograph of E12.5 mouse neurospheres transfected with the REST(810-910)-IRES-GFP (green) and stained for neural progenitor marker SOX2 (blue).

FIG. 7B is an image of a Western blot showing endogenous REST protein levels in E12.5 neurospheres after transfection. Lane 1, empty vector; lane 2, control (595-694) peptide; lane 3, REST(810-910) S861/864A peptide; lane 4, REST(810-910) WT peptide; lane 5, empty vector with MG132 treated for 4 h. The blot was probed with anti-mouse-REST antibody and anti-tubulin (loading control) antibodies.

FIG. 7C is a histogram summarizing the experiment shown in FIG. 7B. There is a significant difference between REST (810-910) compared to control (595-694) peptide (n=5, p=0.0021) and REST(810-910) compared to S861/864A REST(810-910) (n=5, p=0.046). The difference between MG132 and REST(810-910) is not significant (n=5, p=0.7996, Sidak's multiple comparison test, error bars represent 95% confidence interval).

FIG. 8A and FIG. 8B collectively show that expression of the REST (810-910) peptide inhibits neuronal differentiation.

FIG. 8A is a photomicrograph of representative immuno-labeled E12.5 neurospheres transfected with REST (810-910)- or control peptide-IRES-GFP, sorted, and then allowed to differentiate for 10 d in culture. White, DAPI nuclei; Red, MAP2 antibody.

FIG. 8B is a histogram showing that neurospheres expressing REST (810-910) peptide have fewer MAP2-positive cells than cells transfected with control peptide. Results are from three different transfections. Fields were selected from multiple places on the cover slip, number of cells indicated on bars (p<0.0001, unpaired t test).

FIGS. 9A and 9B collectively show the characterization of anti-REST phospho-S861/S864 polyclonal antibody. HEK cells were transfected with FLAG-REST or the FLAG-REST S861A/S864A mutant cDNAs. Cells were treated with 10 uM MG132 prior to collection at 48 h post-transfection.

FIG. 9A is an image of a blot probed with anti-REST p73 directed against the N-terminal region of REST.

FIG. 9B is an image of a blot probed with anti Phospho-REST S861/S864 antibody. Lane 1) WT REST treated with lambda phosphatase buffer, Lane 2) WT REST treated with lambda phosphatase (lane 3). Mutant REST S861A/S864A treated with buffer, lane 4 Mutant REST S861A/S864A treated with phosphatase either condition.

FIG. 10 is an image of a gel that shows REST (810-910) and GFP cDNA co-transfected into HEK cells in the presence and absence of PD184352. Note loss of band representing the doubly phosphorylated peptide and a reciprocal increase in the band representing the non-phosphorylated peptide. Gels were treated the same as in FIG. 5C.

FIG. 11A is a set of two images that show that exogenous wild type REST (810-910) peptide inhibits cortical neuronal migration in an in vivo measure of differentiation. REST protein lacking S861/864 (negative control) and wild-type REST (810-910) peptide (experimental) were expressed in the lateral ventrical of the cortex of rat embryos (E16) by injection and in utero electroporation of the respective cDNA into neural stem cells. The embryos were then allowed to gestate for 48 hours (E18) prior to harvesting. Panel 1 and 2 are representative E18 cortical brain slices expressing REST protein lacking S861/864 (negative control) and wild-type REST protein (experimental). Panel 1, REST protein lacking S861/864 does not interfere with neural progenitor cell migration from the sub ventricular zone (SVZ) (Box1) toward the cortical plate (CP) (Boxes 3 and 4). Panel 2, wild-type REST (810-910) peptide significantly retards migration of neural progenitor cells from the SVZ (Box1) toward the CP (Boxes 3 and 4).

FIG. 11B is a plot showing that the effect of wild type REST (810-910) peptide on neuronal migration is quantitative. An analysis of 6 embryos and 18 cortical slices per injection shows significantly less migration to the CP in embryos treated with wild-type REST (810-910) peptide. No significant difference in neural stem cell migration between the control REST protein lacking S861/864 (negative control) and the wild-type REST (810-910) peptide (experimental) in the SVZ and the intermediate zone (IZ) (Boxes 1 and 2). The wild-type REST (810-910) peptide (experimental) significantly retards migration toward the CP as compared to the REST protein lacking S861/864 (negative control), (Boxes 3 and 4). Error bars represent standard deviation, (**P<0.001, *P<0.01 unpaired t test).

### SEQUENCE LISTING

SEQ ID NO: 1 is the amino acid sequence of the 5-mer REST 861-865 Pin1 binding motif: sppsp.

SEQ ID NO: 2 is the amino acid sequence of a 100-mer peptide that includes SEQ ID NO: 1.

SEQ ID NO: 3 is the amino acid sequence of a 100-mer peptide identical to SEQ ID NO: 2 except that serine 861 and 864 mutated to alanine.

SEQ ID NO: 4 is the sequence of a synthetic peptide used to immunize rabbits to produce a polyclonal antibody that recognizes REST phosphorylated at serines 861 and 864. Both serines are phosphorylated in the peptide.

SEQ ID NO: 5 is amino acid sequence of human CTDSP1.

### DETAILED DESCRIPTION

### I—Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), 5 The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In addition, the materials, methods, and examples are illustrative only and not intended to be limiting. In order to facilitate review of the various embodiments of the disclosure, the following explanations of specific terms are provided:

Antibody: A polypeptide including at least a light chain or heavy chain immunoglobulin variable region which specifically recognizes and binds an epitope of an antigen (such as S861/S864 phosphorylated REST) or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody. In some examples, antibodies of the present disclosure include those that are specific for REST phosphorylated at serine 861 and serine 864.

The term antibody includes intact immunoglobulins, as well the variants and portions thereof, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies, heteroconjugate antibodies (such as bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997. The term antibody also includes polyclonal antisera comprising a library of antibodies raised against an antigen in an individual or set of individuals as well as monoclonal antibodies in which an antibody of a single sequence is expressed by an immortalized cell line.

Binding or stable binding: An association between two substances or molecules, such as the association of an antibody with a peptide, nucleic acid to another nucleic acid, or the association of a protein with another protein or nucleic acid molecule. Binding can be detected by any procedure known to one skilled in the art, such as by physical or functional properties of the antibody antigen complex. For example, binding can be detected functionally by determining whether binding has an observable effect upon a biosynthetic process such as expression of a gene, DNA replication, transcription, translation, and the like. Or binding can be detected physically through gel shift assays, changes in surface plasmon resonance, or coimmunoprecipitation.

Contacting: Placement in direct physical association, including both a solid and liquid form. Contacting can occur in vitro with isolated cells or tissue or in vivo by administering to a subject.

Mass spectrometry: A method wherein a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation.

Phospho-peptide or phospho-protein: A peptide or protein in which one or more phosphate moieties are covalently linked to amino acid residues or amino acid analogs. A peptide can be phosphorylated at multiple or single sites. Sometimes it is desirable for the phospho-peptide to be phosphorylated at one site regardless of the presence of multiple potential phosphorylation sites. The transfer of a phosphate to a peptide is accomplished by a kinase exhibiting kinase activity. The removal of a phosphate from a peptide is accomplished by a phosphatase exhibiting phosphatase activity.

Polypeptide: Any chain of amino acids, regardless of length or posttranslational modification (such as glycosylation, methylation, ubiquitination, phosphorylation, or the like). Examples of polypeptides include REST, a 100mer peptide thereof, or CTDSP1. “Polypeptide” is used interchangeably with “peptide” or “protein”, and is used to refer to a polymer of amino acid residues. A “residue” refers to an amino acid or amino acid mimetic incorporated in a polypeptide by an amide bond or amide bond mimetic.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Small molecule: A molecule, typically with a molecular weight less than about 1000 Daltons, or in some embodiments, less than about 500 Daltons, wherein the molecule is capable of modulating, to some measurable extent, an activity of a target molecule such as inhibiting the activity of a molecule that promotes REST stability particularly a REST phosphatase such as CTDSP1.

Specific Binding Agent: A protein-specific binding agent binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

### II—Screening Methods

Disclosed herein are methods of selecting test compounds that promote REST degradation. These methods involve, adding a test compound, a peptide comprising an SPPSP sequence equivalent to SEQ ID NO: 1 herein; and an active CTDSP1 protein (represented by SEQ ID NO: 5 herein) both serines of SEQ ID NO: 1 in the peptide are phosphorylated. If the test compound results in the maintenance of phosphorylation of the serines of SEQ ID NO: 1, then the test compound is a compound that promotes REST degradation.

The test compound can be any compound including a small molecule, nucleic acid, protein, antibody, or any other composition that has or could have biological activity. In some examples, the test compound is a positive control known to promote REST degradation, potentially through the inhibition of CTDSP1. One example is an antibody that blocks the activity of CTDSP1. In further examples the test compound is a negative control known to have no effects on REST or CTDSP1. An example of a negative control is a vehicle or buffer in which the test compounds are added to the mixture.

Adding compounds to a mixture can be performed in any order. The mixture can also comprise additives not in the claim such as buffer salts, preservatives, relatively inert proteins such as albumins, fluorescent compounds, and other additives.

In one example, a composition that fluoresces in the presence of free phosphate is added to the mixture. A test compound that does not promote REST degradation or a negative control test compound will result in the phosphates conjugated to the serines of the peptide being removed by CTDSP1, resulting in an increase in free phosphate and increased fluorescence in the mixture. A test compound that does promote REST degradation will maintain phosphates on the serines of the peptides, resulting in a smaller increase in free phosphate and therefore less fluorescence relative to the negative control.

Other methods of selecting a test compound that promotes REST degradation involve adding the test compound to the mixture, adding the peptide comprising SEQ ID NO: 1 to the mixture, wherein both serines of SEQ ID NO: 1 are phosphorylated; adding an antibody that specifically binds the peptide when both serines are phosphorylated and does not bind the peptide when both serines are dephosphorylated to the mixture, and adding an active CTDSP1 protein to the mixture.

The antibody can be any antibody created for this purpose including a monoclonal or polyclonal antibody. Methods of generating such an antibody can be performed by one of skill in the art in light of this disclosure. Some such methods involve immunizing a subject with a peptide of SEQ ID NO: 4 herein.

The peptide can be any peptide comprising SEQ ID NO: 1 including peptides at least 50%, at least 60%, at least 70%, at least 80%, at least 90%, at least 95%, or 100% identical to SEQ ID NO: 2 or peptides at least 50%, at least 60%, at least 70%, at least 80%, at least 90%, at least 95%, or 100% identical to full-length wild type REST or any fragment of REST that is degraded upon dephosphorylation of the serines of SEQ ID NO: 1.

### EXAMPLES

### Example 1

### Phosphorylation Status of Serines 861/864 Affects REST Protein Stability

Because REST is a relatively large protein (predicted size 122 kDa), it was hypothesized that multiple phosphorylation sites impact its stability. FLAG-tagged REST protein stably expressed in PC12 cells were treated with the inhibitor MG132 (Ballas N et al, 2005 supra). REST is turned over rapidly in PC12 cells unless MG132 is added to stabilize the turnover. Mass spectrometry revealed a total of 14 phosphorylated residues including phosphorylation at serine 1013 as previously described in the literature (Guardavaccaro et al, 2008 supra, Westbrook et al, 2008 supra).

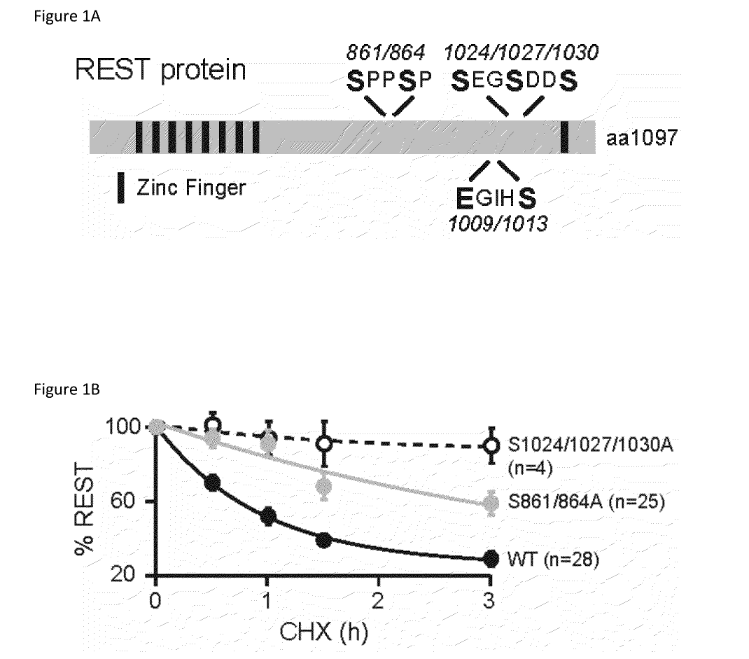

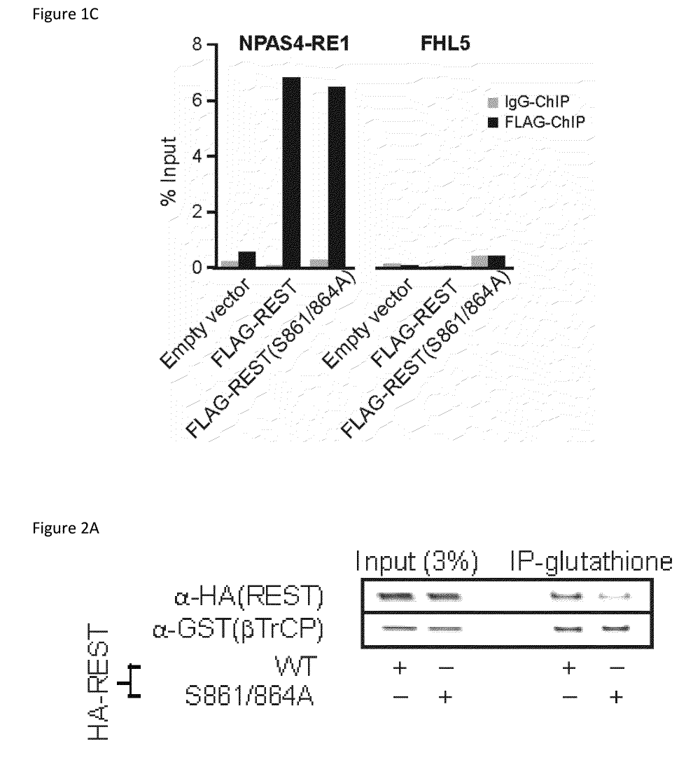

Two amino acids in particular, S861 and S864, stood out due to their high frequency of phosphorylation (FIG. 1A). The S861/S864 site was further analyzed in HEK293T cells due to greater ease of transfection of HEK293T cells relative to PC12 cells in transient transfection assays. Mutation of S861 and S864 to alanines, in the context of full-length REST, prolonged the half-life of REST protein in HEK cells treated with cyclohexamide (CHX), similar to the effect of mutations within the two C-terminal degrons (S1024/S1027/S1030) (Westbrook et al, 2008 supra) (FIG. 1B). Mutation of other residues in the C-terminus did not significantly affect REST stability.

Mutation of S861/S864 to alanine did not perturb in vivo binding of REST to the RE1 consensus binding site located within the first intron of the NPAS4 transcription factor target gene (Bersten D C et al, Biochim Biophys Acta 1839, 13-24 (2014); incorporated by reference herein) These results demonstrated that the S861A/S864A double mutant of REST did not impair nuclear import and localization to genomic targets relative to the unmutated wild type (WT) protein (FIG. 1C). So without disruption of normal REST activity, the prevention of phosphorylation at S861 and S864 made the REST protein more physiologically stable.

### Example 2

### Phosphorylation of Serines 861/864 Regulates βTrCP Binding to REST

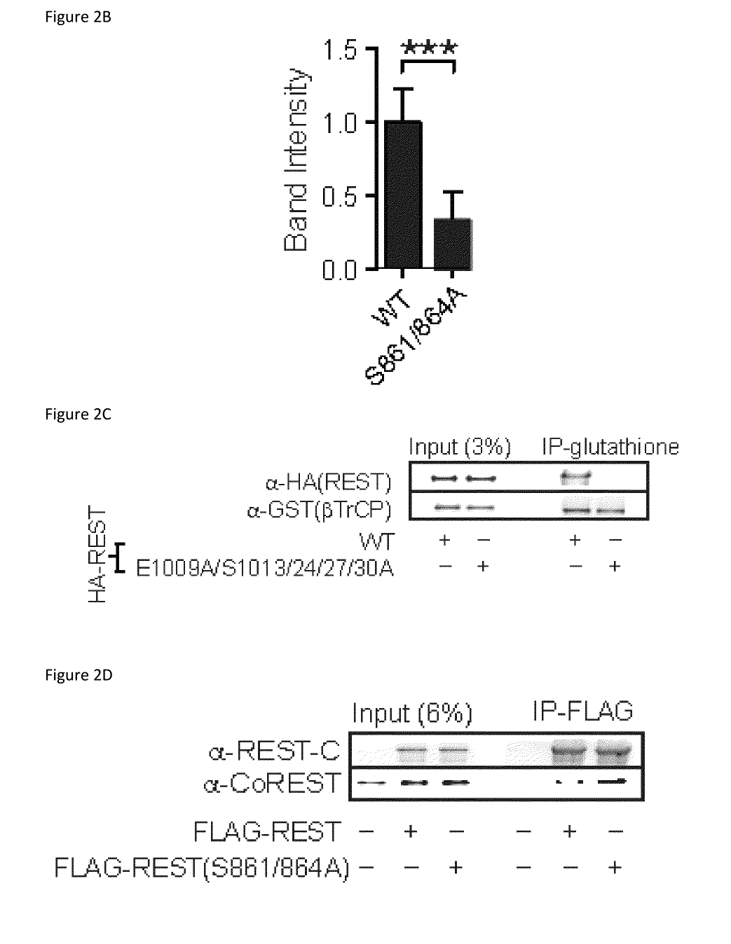

It is known that that binding of βTrCP to REST is involved in REST degradation. βTrCP specifically binds to serines at position S1024, 1027, and 1030 (Westbrook et al, 2008 supra). To assess whether the S861/S864 was involved in βTrCP-binding, HEK cells were cotransfected with cDNAs encoding a βTrCP-GST fusion as well as HA-tagged versions of unmutated wild type (WT) REST S861/S864 or mutant S861A/S864A REST cDNA. After 48 hours, cell extracts were precipitated with glutathione beads and resolved on SDS gels, followed by Western blotting for HA and GST epitopes. Unmutated REST was present in the GST-βTrCP precipitates, as predicted, but REST with S861A/S864A mutations was barely detectable, suggesting either that the mutations disrupted βTrCP binding to this site, or that the mutations at this site prevented binding of βTrCP binding to the other degrons due to a change in REST conformation (FIG. 2A, B).

To distinguish between these two possibilities, the converse experiment was performed: and GST-βTrCP fusion cDNA was co-transfected with REST cDNAs encoding mutations E1009A-51013A/S1024A/S1027A/S1030A, but unmutated S861/S864. This REST protein was not detected in the GST-βTrCP pull-down, indicating that βTrCP does not bind to the S861/S864 site directly in the absence of other sites in the C-terminal region of REST (FIG. 2C). Taken together, these results suggest that the S861/S864 site does not interact directly with βTrCP, but that phosphorylation at this site is required for binding of βTrCP to the downstream degrons.

A phosphorylation-dependent change in the conformation of the C-terminus of REST may also influence binding of REST to other proteins, such as its co-repressors. To test this idea, unmutated REST or S861A/S864A REST was transfected into HEK cells and changes in the amount of CoREST in REST complexes were observed. Preventing phosphorylation increased interaction with the corepressor, CoREST, in contrast to its reduced binding to βTrCP (FIG. 2D).

### Example 3

### REST-GFP Fusion Peptides can be Used to Monitor Phosphorylation Status of Serines 861/864 in REST

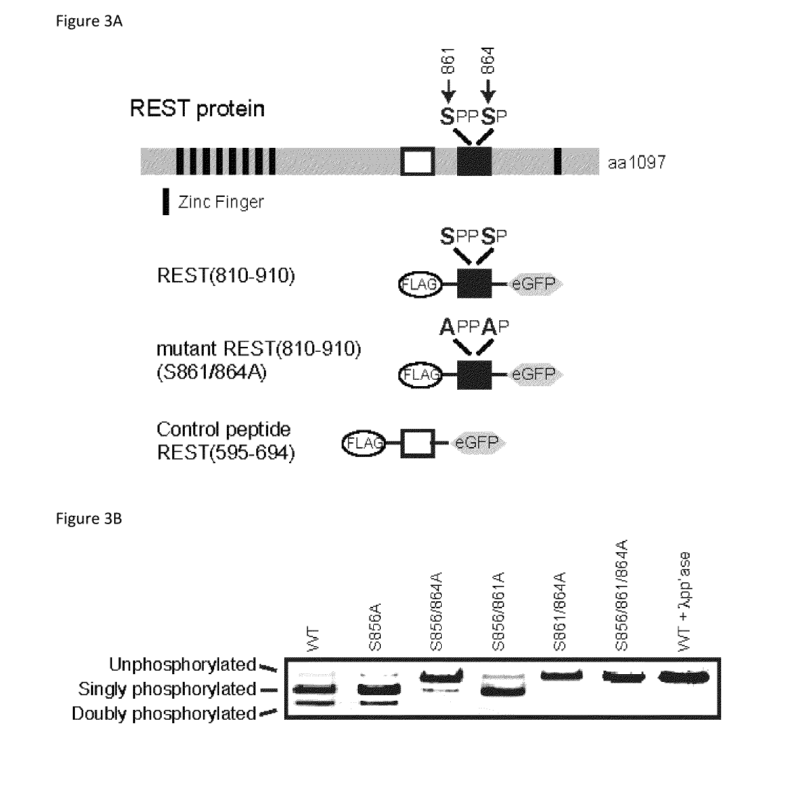

To monitor phosphorylation status of specific REST sites under different circumstances, a cDNA reporter construct that expresses a peptide encoding amino acids 810-to-910 of REST (FIG. 3A) was constructed. Mutant REST (810-910) cDNAs encoding alanine mutations at S861, S864 and at an adjacent site, S856 were also constructed. S856 was minimally phosphorylated in the mass spectrometric analysis described above. A cDNA for a control REST peptide (aa595-694) that does not contain any known phosphorylated residues was also constructed. The constructs also comprise regions that encode N-terminal FLAG and C-terminal eGFP tags (FIG. 3A). These constructs were transfected into HEK cells and expressed. Extracts from the cells were resolved on native gels 24 hours after transfection. The expressed peptides were detected by in-gel GFP fluorescence.

The phosphorylation state of the expressed REST peptides can be visualized by differential migration in native gels due to charge differences. The unmutated S861/S864 REST 810-910 peptide (FIG. 3B, lane 1) migrated predominantly as two distinct bands that were not present upon treatment with lambda phosphatase (FIG. 3B, lane 7). Phosphatase treatment resulted in a single, slower migrating species, which was understood to be a non-phosphorylated peptide because it comigrated with the very low abundance species in the untreated sample (top band, lane 1). The identification of the doubly phosphorylated species was based upon the mobility of an unmutated peptide containing unmutated S861/864 (FIG. 3B, lane 2, S856A), which was not present in samples expressing peptides containing S861A or S864A single mutations (lanes 3 and 4, respectively). The peptides with S861A and S864A double mutations (FIG. 3B, lane 5) showed primarily the non-phosphorylated species, whether or not the S856A mutation was also included (FIG. 3B, lane 6). These results suggest that S861 and S864 are major sites of phosphorylation in this cellular context and establish the utility of the reporter peptide to indicate the phosphorylation status of REST.

### Example 4

### Peptidyl-Prolyl Cis-Trans Isomerase (Pin1) Activity at Serines 861/864 Promotes REST Degradation

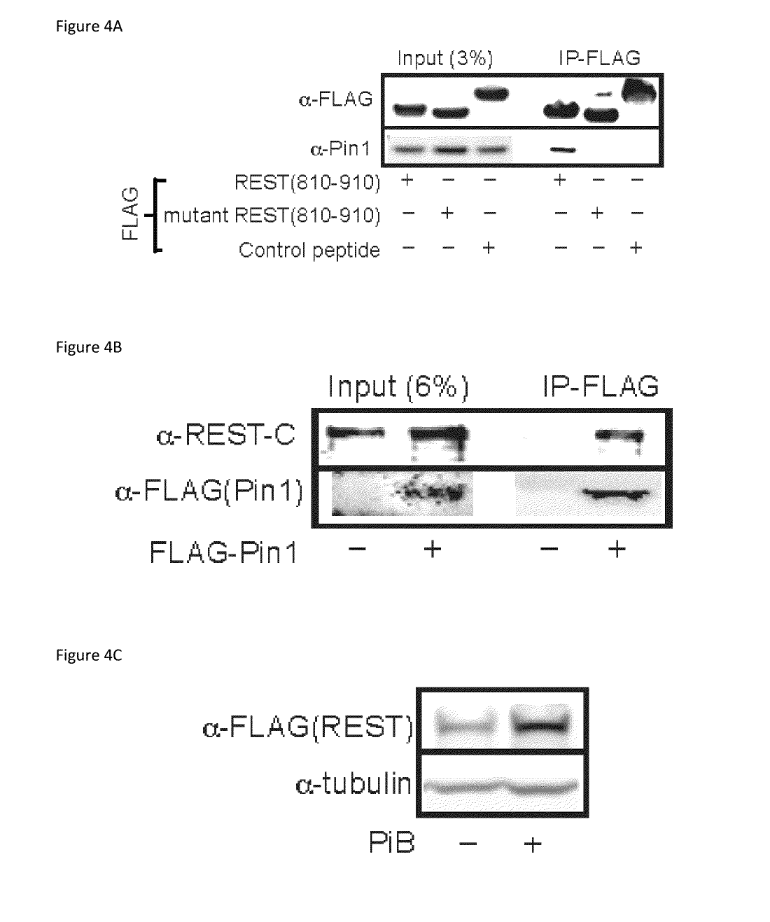

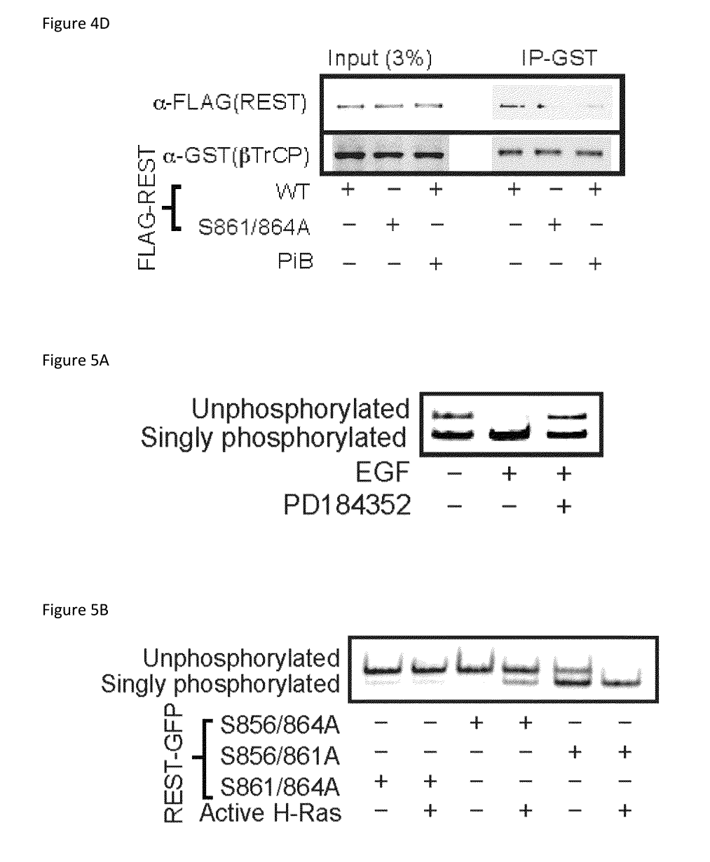

The SPPSP site from amino acid 861-865 in REST (SEQ ID NO: 1) forms a binding motif for Pin1 (Lu P J et al, J Biol Chem 277, 2381-2384 (2002); incorporated by reference herein) and it is likely that Pin1 interacts with REST at this site. To confirm this, HEK cells were transfected with FLAG-Pin1 and cDNAs encoding the 100 amino acid peptides described above containing an S861/S864 or an S861A/S864A sites. Pin1 was present in immunocomplexes of unmutated REST (810-910) reporter peptides, but not phosphorylation-resistant mutant S861A/S864A peptides (FIG. 4A). Full-length REST was also co-purified with Pin1 immunocomplexes after transfection of both cDNAs in HEK cells (FIG. 4B) and inhibition of Pin1 activity with the proteasomal inhibitor PiB (which in turn inhibited REST degradation) (FIG. 4C). Further, βTrCP binding to stabilized (MG132-treated) WT REST was also disrupted by PiB treatment, similar to the effect of the S861A/S864A double mutant peptide that prevents phosphorylation at this degron (FIG. 4D).

### Example 5

### REST Stability is Regulated, at Least in Part, Through ERK Phosphorylation of Serines 861/864

Pin1 binding to prolines requires phosphorylation of an adjacent serine or threonine (Lu P J et al, 2002 supra). Because S861/S864 are highly predicted target sites for the proline-directed kinases ERK1 and 2, it was hypothesized that ERK and its upstream activators, EGF and Ras, would promote phosphorylation at these sites in REST.

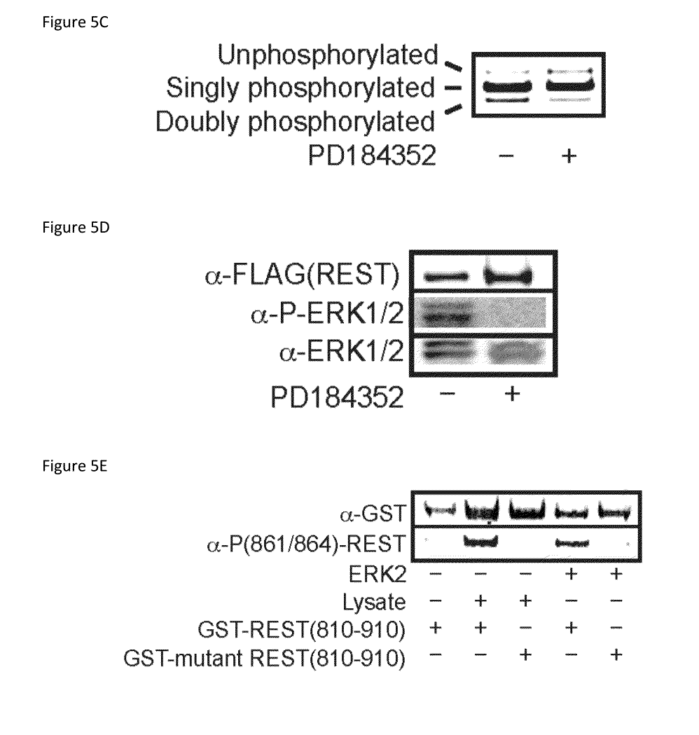

The reporter peptides were used to test the effects of EGF treatment on REST phosphorylation in HEK cells. It was observed that treatment with recombinant EGF alone caused a mobility shift in the S864 peptide. This mobility shift is indicative of phosphorylation (FIG. 5A). The mobility shift was prevented by prior treatment with PD184352, a MEK (ERK-activation) inhibitor (Ley R et al, J Biol Chem 278, 18811-18816 (2003); incorporated by reference herein). Next, it was assessed whether a constitutively active Ras could induce phosphorylation of either S861 or S864 in HEK cells. Constructs to express three additional reporter peptides were generated each containing with a single potentially phosphorylated serine. These peptides can be described as having the mutations S856/S861A/S864A; S856A/S861/S864A; and S856A/S861A/S864. These were introduced into HEK cells and monitored by in-gel GFP fluorescence. Only the peptides with individual unmutated S861 or S864, but not the S856/S861A/S864A peptide, underwent a mobility shift indicative of Ras-induced phosphorylation (FIG. 5B).

The improved stability of full-length REST protein containing alanine mutations at residues S861 and S864 (FIG. 1B) suggests that endogenous kinase activity in HEK cells phosphorylates these residues. To provide further support for this, unmutated REST (810-910) reporter cDNA was transfected into HEK cells and the peptide expressed in the presence and absence of the MEK/ERK inhibitor PD184352. Treatment with PD184352 caused a modest loss from the band at the position representing unmutated S861/S864 peptide and a reciprocal modest increase in the species representing unphosphorylated peptide (FIG. 5C). This result predicts that ERK inhibition should also stabilize REST. Accordingly, HEK cells transfected with FLAG-REST cDNA were treated with PD184352 for 30 min and the extracts were analyzed by Western blot. The inhibitor treatment resulted in increased REST protein levels compared to treatment with vehicle (FIG. 5D).

### Example 6

### C-Terminal Domain Small Phosphatase (CTDSP1) Activity at Serines 861/864 Stabilizes REST Protein

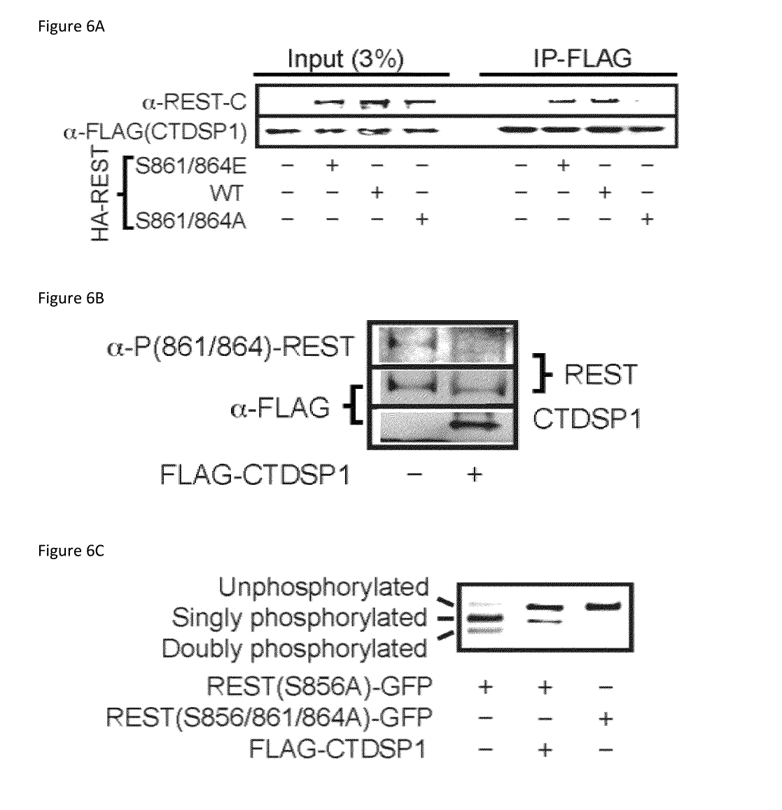

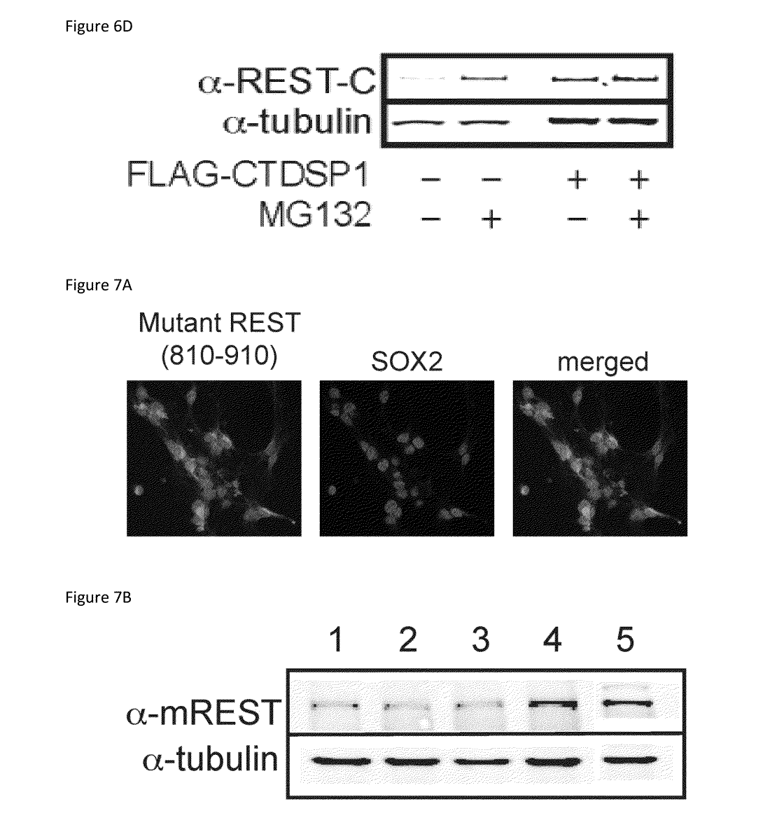

Protein stability is often regulated by a balance between kinase and phosphatase activities. CTDSP1 was first identified as a nuclear phosphatase targeting the C-terminus of RNA polymerase II (R HR et al, BMB Rep 47, 192-196 (2014); Feng Y et al, Biochem Biophys Res Commun 397, 355-360 (2010); and Zhang Y et al, Mol Cell 24, 759-770 (2006); all of which are incorporated by reference herein). Subsequently it was implicated in REST repression of neuronal gene expression in P19 embryonal carcinoma cells that have the capacity to differentiate into neurons (Visvanathan J et al, Genes Dev 21, 744-749 (2007); incorporated by reference herein). To test whether this effect on neuronal gene repression might result from CTDSP1 influence on REST protein stability, HEK cells were transfected with FLAG-CTDSP1 along with full-length REST cDNAs encoding either unmutated S861/S864, or glutamate (S861E/S864E) or alanine (S861A/S864A) substitutions at these sites (FIG. 6A). REST was detected in FLAG-immunocomplexes only when the site was unmutated or double-substituted with glutamate, a phosphorylation-mimic.

The S861A/S864A mutant did not form complexes with CTDSP1. Using a REST phospho-specific polyclonal antibody, it was shown that exogenously expressed FLAG-CTDSP1 dephosphorylated these sites in full-length REST (FIG. 6B). Dephosphorylation by CTDSP1 was also observed in the REST reporter peptides as detected by in-gel fluorescence (FIG. 6C). The dephosphorylation by CTDSP1 resulted in REST stabilization, which was not increased further by treatment with the proteasomal inhibitor MG132 (FIG. 6D).

### Example 7

### Expression of the REST (810-910) Peptide Stabilizes Endogenous REST

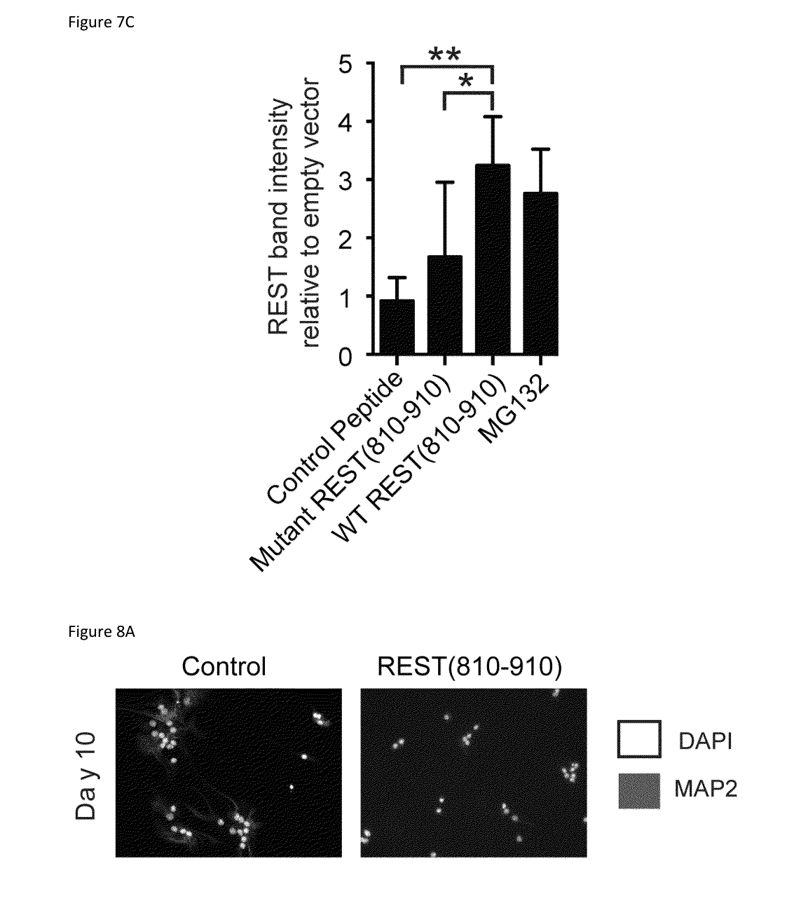

The Examples above used the REST (810-910) reporter as a surrogate substrate for signaling molecules that target S861/S864 on full-length REST. The peptide can also be used as a signaling decoy to stabilize full-length REST protein in mouse neural progenitors, where REST turnover is enhanced in preparation for terminal neuronal differentiation (Ballas N et al, 2005 supra). Cultured E12.5 cortical neurospheres were Sox2-positive and remained Sox2-positive after expression of the mutant REST (810-910) peptide for 24 h (FIG. 7A) demonstrating that transfection does not alter the neural progenitor state of the culture. However, transfection with cDNA encoding WT REST (810-910) peptide (FIG. 7B, lane 4; FIG. 7C) significantly stabilized endogenous full-length REST protein compared to mutant S861A/S864A or other negative control peptides (FIG. 7B, lanes 2 and 3, respectively; FIG. 7C). MG132 did not stabilize the Rest protein further (FIG. 7B, lane 5; FIG. 7C).

### Example 8

### Expression of REST (810-910) Inhibits Neuronal Differentiation

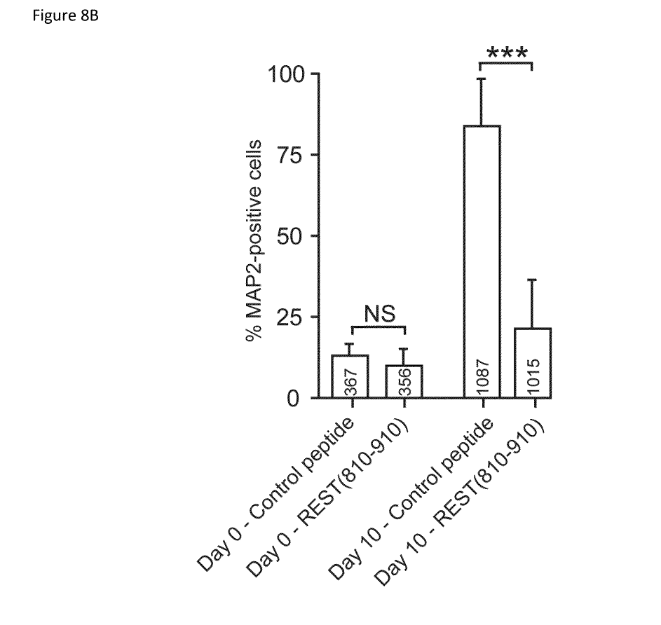

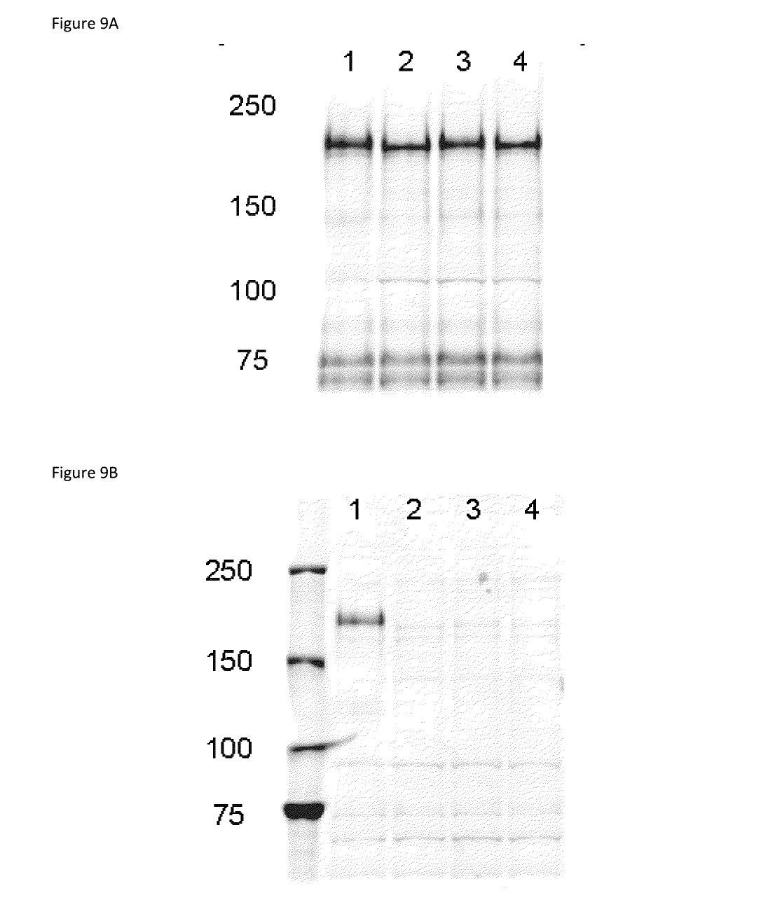

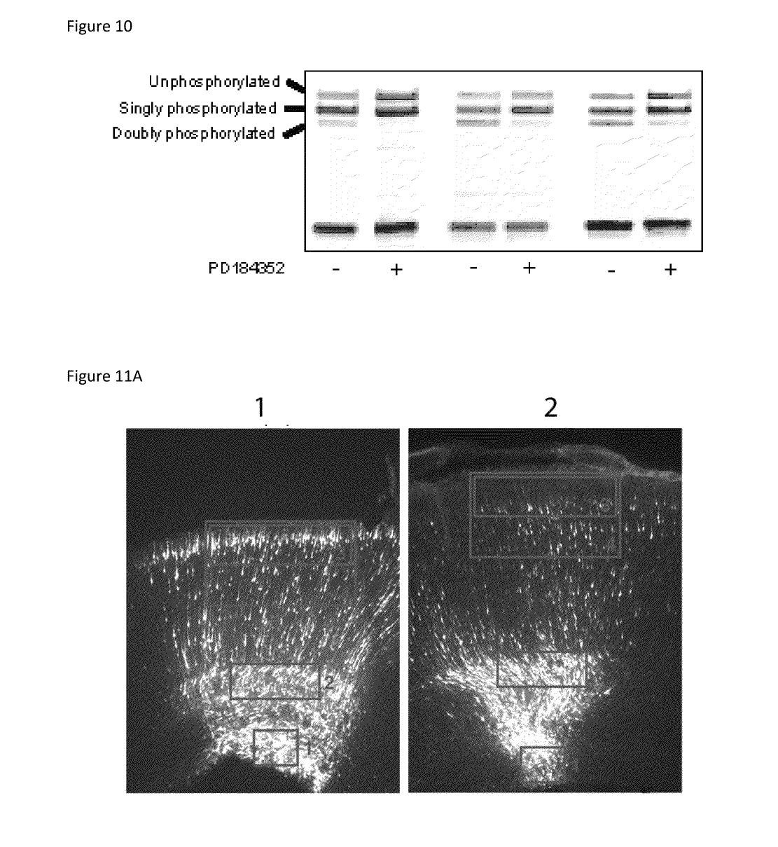

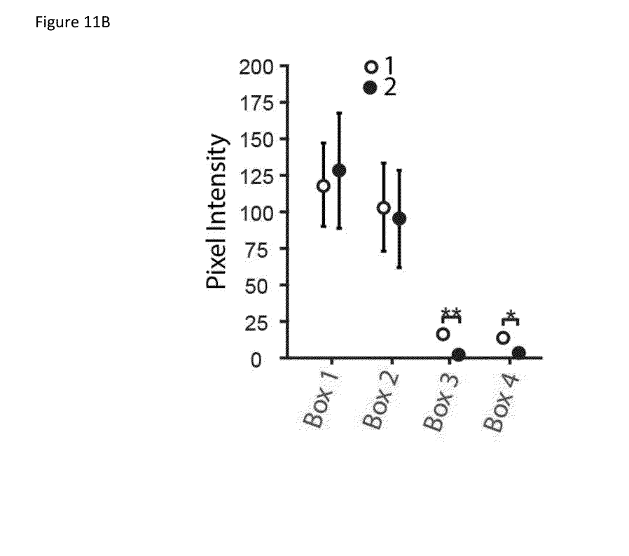

Previous studies indicate that persistent REST expression in progenitors inhibits neuronal differentiation. Therefore, it was assessed whether stabilization of REST, via expression of the unmutated REST (810-910) peptide in neural progenitors, would also hinder neuronal differentiation. Mouse neurosphere cultures were immunostained for Microtubule Associated Peptide 2 (MAP2), a marker of mature neurons, at days 0 and 10 post-transfection. On day 0 there was negligible MAP2 signal and no significant difference between the two conditions. At day 10, there was significantly less MAP2 expression in neurospheres transfected with REST (810-910) compared to the REST (595-694) control peptide (FIGS. 8A and 8B).

### Example 9

### Experimental Methods

Plasmids:

HA-REST and mutant REST (E1009A/S1013A) cDNAs were synthesized as described in (Guardavaccaro et al 2008 supra). HA-REST and REST mutations (S1024/1027/1030A, E1009/S1013/1024/1027/1030A, S861/864A and S861/864E) were generated by overlap extension PCR. HA was replaced with the FLAG® epitope for some experiments. For REST peptide reporter constructs, selected regions of REST were PCR-amplified with flanking restriction sites to permit insertion along with FLAG linker into pEGFP-N1 (Clontech). WT and mutant REST regions were also cloned into pCA-IRES-GFP as described in (Matsuda T and Cepko C L, Proc Natl Acad Sci USA 101, 16-22 (2004); incorporated by reference herein) for expression in neural progenitors or into pGEX-3X (GE Healthcare) for bacterial expression. All constructs were confirmed by DNA sequence analysis. GST-βTrCP and dnCul1 (aa1-452) are described in (Jin J et al, Genes Dev 17, 3062-3074 (2003); Jin J et al, Methods Enzymol 399, 287-309 (2005); both of which are incorporated by reference herein. Human Pin1 and FLAG-Xenopus Pin1 are described in Winkler K E et al, Science 287, 1644-1647 (2000); and Stukenberg P T and Kirschner M W, Mol Cell 7, 1071-1083 (2001); both of which are incorporated by reference herein.

Transient Transfections and Cell Culture:

HEK293T cells were transfected using Lipofectamine 2000 (Life Technologies) following the manufacturer's instructions. PC12 tet, PC12 tet-on FLAG-REST, and HEK293T cell lines were maintained as described in Ballas N et al 2001 supra. Neurospheres were isolated from E12.5 mouse cortices and cultured as described in Reynolds B A and Weiss S, Science 255, 1707-1710 (1992); incorporated by reference herein in neurobasal media supplemented with 2 mM L-glutamine, 100 U/mL penicillin, 100 mg/mL streptomycin, B27 supplement, 20 ng/mL EGF, and 10 ng/mL FGF-2, all from Life Technologies. Neurospheres were passaged every 3-4 days using Accutase® (Sigma). For stability analysis, neurospheres (passage 4/5) were dissociated and 0.5×10⁶ cells were transfected 3 μg of plasmid using Lipofectamine 2000® (Life Technologies). For differentiation analysis, neurospheres (passage 4/5) were dissociated, transfected with GFP constructs, and 48 h later they were dissociated again and sorted for GFP. 0.2×10⁶ GFP-positive neurospheres were plated on a coverslip pre-coated with laminin (20 mg/ml, BD Bioscience) and poly-D-lysine (200 mg/ml, Sigma) and incubated in neurobasal medium containing 0.5% FBS and lacking EGF and FGF-2.

Mass Spectrometry:

Ten 150 mm plates of PC12 tet-on FLAG-REST cells were treated with 10 mM MG132 for 5 h prior to immunoprecipitation with FLAG antibody (M2 agarose; Sigma). Samples were reduced with DTT, alkylated with IAA (iodoacetamide) and digested overnight with the following enzymatic conditions: Trypsin and GluC, GluC only, and GluC+Asp-N. For analysis on the mass spectrometer each protein digest was analyzed by LC-MS using an Agilent 1100 Series® Capillary LC system (Agilent Technolgies Inc, Santa Clara, Calif.) and an LTQ® linear ion trap mass spectrometer (ThermoFisher, San Jose, Calif.). Electrospray ionization was performed with an ion max source fitted with a 34 gauge metal needle (ThermoFisher, cat. no. 97144-20040) and 2.7 kV source voltage. Samples were applied at 20 μL/min to a trap cartridge (Michrom BioResources, Inc, Auburn, Calif.), and then switched onto a 0.5×250 mm Zorbax® SB-C18 column with 5 mM particles (Agilent Technologies) using a mobile phase containing 0.1% formic acid, 7-30% acetonitrile gradient over 95 min, and 10 μL/min flow rate.

Data-dependent collection of MS/MS spectra used the dynamic exclusion feature of the instrument's control software (repeat count equal to 1, exclusion list size of 50, exclusion duration of 30 sec, and exclusion mass width of −1 to +4) to obtain MS/MS spectra of the three most abundant parent ions (minimum signal of 10,000) following each survey scan from m/z 400-2000. The tune file was configured with no averaging of microscans, a maximum MS1 inject time of 200 msec, a maximum MS2 inject time of 100 msec, and automatic gain control targets of 3×10⁴ in MS1 mode and 1×10⁴ in MS2 mode.

Interaction Studies:

GST pull-down experiments and Western blots were performed as described in Westbrook et al, 2008 supra.

Chromatin Immunoprecipitation:

ChIP analyses with REST were performed as described in Ballas et al, 2005 supra. Briefly, 10′ HEK293T cells transfected with constructs expressing versions of FLAG-REST were crosslinked in 1% formaldehyde for 10 min and quenched in 0.125 M glycine for 5 min at room temperature. Cell lysates were sonicated to shear chromatin fragments to a size range of ˜100-750 bp. Chromatin was purified from these lysates using either 5 μg of anti-FLAG M2 (Sigma) or non-specific mouse IgG. Quantitative real-time PCR (qRT-PCR) measured relative quantities of co-immunoprecipitated NPAS4 and FHL5 genomic regions. Primer sequences (5′->3′) used for qRT-PCR were; NPAS4-F: CCTGAGCCTAGGGGAACATAG, NPAS4-R: CATGGACAGAGCCATACACG, FHL5-F: ACAGGTGCCAAGTTTATCTGC and FHL5-R TACCCACCAAGGAGACAGAG.

Co-Immunoprecipitation and Western Blot Analysis:

Whole cell lysates were prepared and immunoprecipitated following the procedures in Ballas N et al, 2001 supra and Nesti E et al, Mol Biol Cell 15, 4073-4088 (2004); incorporated by reference herein. Western blots were performed by standard procedures using antibodies described below and analyzed using anti IgG conjugated to infrared dyes (ThermoFisher) on an Odyssey® infrared fluorescence imager (LiCor). For detecting phosphorylated REST at S861/S864, cell extracts were prepared lacking phosphatase inhibitors. For phosphatase treatment, 75 μg of total protein was treated with 600 U lambda phosphatase (New England Biolabs) or with buffer alone at 30° C. for 1 h, according to the manufacturer's protocol. Thirty-five μg of treated protein were resolved by SDS-PAGE and Western blotted with the non-phospho-depleted anti REST phospho antibody (rabbit #101) at 1:500 in 3% BSA/1×TBS-0.5% Tween-20® overnight at 4° C., followed by anti-rabbit IgG-IR680 conjugated secondary antibody (Thermo Fisher) at 1:15000 in 5% milk/TBS-Tween for 1 hour at room temp. Infrared fluorescence was detected on the Odyssey® imager (LiCor) in the 700 nm channel. The blot was re-probed with anti-REST p73 antibody (Chong et al 1995 supra) at 1:1000, followed by anti-rabbit IgG-IR800 conjugated secondary antibody (Thermo Fisher) and detection in the 800 nm channel.

In Vitro Binding Assay:

Bacterially expressed GST-REST peptides were purified by glutathione agarose affinity chromatography (Hattan D et al, J Biol Chem 277, 38596-38606 (2002); incorporated by reference herein. ERK2 in vitro kinase analysis with purified protein was performed as described Li Y et al, J Biol Chem 288, 27646-27657 (2013); incorporated by reference herein.

Antibodies and Chemicals:

REST-C (Ballas et al, 2005 supra) and CoREST antibodies (Andres M E et al, Proc Natl Acad Sci USA 96, 9873-9878 (1999); incorporated by reference herein). HA-probe (F-7) (Santa Cruz Biotechnology); anti-FLAG M2 (Sigma); anti-GST, (Thermo Fisher); anti-phospho-ERK1/2 (Cell Signaling); anti-ERK1/2 (Transduction Laboratories); anti-alpha-tubulin (DSHB, University of Iowa). Cyclohexamide solution (Sigma); MG132 (Calbiochem); PiB (Sigma); PD184352 (LC Laboratories).

Imaging:

Detection of in gel fluorescence on native 10.5%-14% acrylamide gels (Bio-Rad Cat #345-9949) was performed using the Typhoon imaging system by GE Healthcare Life Science.

Preparation of Anti-Mouse REST Polyclonal Antibody #095:

Rabbits were immunized by Covance Research Products (Denver, Pa.) with GST-mouse REST (amino acids 889-1035) peptide, purified by glutathione-agarose affinity chromatography from bacterially expressed lysate. Polyclonal antiserum obtained from a single rabbit #095 was depleted of anti-GST epitopes by passage over GST-agarose, followed by affinity chromatography on its antigen coupled to Affigel-10 resin (Bio-Rad) following standard techniques.

Preparation of Anti-REST Phospho-S861/864 Polyclonal Antibody #101:

Rabbits were immunized by Covance Research Products (Denver, Pa.) with a synthetic peptide (CTEDLpSPPpSPPLPK—SEQ ID NO: 4) representing amino acids 857-869 of human REST, including chemically phosphorylated serines 861 and 864, coupled to keyhole limpet hemocyanin. Polyclonal antiserum obtained from a single rabbit #101 was depleted of non-phospho-specific epitopes by several passages over Sulfolink resin (Thermo Pierce) coupled to a cognate synthetic peptide devoid of serine phosphorylations. The unbound fraction of the serum was used without further manipulation.

### Example 10

### Screening Methods for Identifying Compounds that Regulate Activity

Various screening methods for identifying compounds that regulate activity may be used. As one example a screening method could comprise utilizing a high-throughput fluorescence assay to identify molecules that inhibit CTDSP1 activity on the REST short-peptide. For example, CTDSP1, phosphorylated peptides, and compound libraries can be mixed in the presence of a fluorescent free phosphate readout, e.g., using a suitable phosphate binding protein such as Invitrogen phosphate binding protein. In such an approach, inhibitors of CTDSP1 activity may show decreased fluorescence. Such an approach may provide a scalable assay, may be performed in multi-well formats (e.g., 384 well, etc.), and may be amenable to high-throughput and stacking.

In other screening approaches, a Surface Plasmon Resonance (SPR) assay involving, for example, a Biacore system. Such an approach can characterize molecules in terms of specificity of their interactions and may provide sensitive and accurate concentration measurements. This is based on the ability of the biomolecule of interest to interact with a specific binding partner, and may therefore be more informative than generic measurement techniques such as total protein concentration, for example. Such high quality compound screening and characterization may increase productivity and cost-efficiency, facilitate a more confident selection of compounds (high information content), and enable analysis of problem targets with unknown or unstable substrates. Further, such approaches may provide a low consumption of target proteins compared to alternative methods and may efficiently reduce or substantially eliminate false-positives.

One such screening method can utilize a Biacore screen or equivalent assay to detect molecules that inhibit CTDSP1 phosphatase activity on the REST peptide. For example, the phospho antibodies may be fixed to an on-chip for SPR experiments. In such an approach, the reactions consisting of CTDSP1, phosphorylated ligands, and compound libraries can be tested for binding to antibodies. For example, molecules that inhibit CTDSP1 can be determined by phospho antibody binding.

In another example, a screening method can utilize a Biacore screen or equivalent assay to detect molecules that inhibit phosphorylation. For example, the phospho antibodies can be fixed to a chip for SPR experiments. In such an approach, reactions of cell lysate, unphosphorylated ligands, and compound libraries may be tested for binding to antibodies. For example, molecules that inhibit phosphorylation may be determined by loss of phospho antibody binding.

It is to be understood that the examples and/or approaches described herein are exemplary in nature, and that these specific embodiments or examples are not to be considered in a limiting sense, because numerous variations are possible. The subject matter of the present disclosure includes all novel and nonobvious combinations and subcombinations of the various processes, systems and configurations, and other features, functions, acts, and/or properties disclosed herein, as well as any and all equivalents thereof.

1. A method of selecting a test compound that promotes REST degradation, the method comprising:
adding the test compound to a mixture;
adding a polypeptide comprising SEQ ID NO: 1 to the mixture, wherein both serines of SEQ ID NO: 1 are phosphorylated; and
adding a protein at least 70% identical to SEQ ID NO: 5 to the mixture;
wherein a test compound that causes the maintenance of phosphorylation of the serines of SEQ ID NO: 1 is a compound that promotes REST degradation.

2. The method of claim 1 wherein the polypeptide comprises a sequence at least 70% identical to SEQ ID NO: 2.

3. The method of claim 2 wherein the polypeptide consists of SEQ NO: 2.

4. The method of claim 1 further comprising adding a composition that fluoresces in the presence of free phosphate to the mixture and wherein a test compound that generates less fluorescence in a negative control is a compound that promotes REST degradation.

5. A method of selecting a test compound that promotes REST degradation, the method comprising:
adding the test compound to a mixture;
adding a polypeptide comprising SEQ ID NO: 1 to the mixture, wherein both serines of SEQ ID NO: 1 are phosphorylated;
adding an antibody to the mixture, wherein the antibody specifically binds the polypeptide when both serines of SEQ ID NO: 1 are phosphorylated and does not bind the polypeptide when at least one of the serines of SEQ ID NO: 1 is not phosphorylated;
adding a protein at least 70% identical to SEQ ID NO: 5 to the mixture;
detecting the binding of the antibody to the peptide;
wherein a test compound that causes less binding of the antibody to the peptide relative to a negative control is a compound that promotes REST degradation.

6. The method of claim 5 wherein the polypeptide comprises a sequence at least 70% identical to SEQ ID NO: 2.

7. The method of claim 6 wherein the polypeptide consists of SEQ NO: 2.

8. The method of claim 5 wherein the antibody is a polyclonal antibody.

9. The method of claim 5 wherein the antibody is a monoclonal antibody.

10. The method of claim 5 wherein the binding of the antibody to the polypeptide is detected by surface plasmon resonance.

11. A method of stabilizing REST in a cell; the method comprising:
contacting the cell with a peptide comprising SEQ ID NO: 1; wherein the serines of SEQ ID NO: 1 are not phosphorylated, thereby stabilizing REST in the cell.

12. The method of claim 11 wherein the peptide comprises a sequence at least 70% identical to SEQ ID NO: 2.

13. The method of claim 12 wherein the peptide consists of SEQ ID NO: 2.

14. The method of claim 11 wherein the cell comprises an expression vector, the expression vector comprising a first polynucleotide that encodes the peptide and a promoter operably linked to the nucleic acid.

15. The method of claim 11 wherein the cell is in vivo.

16. A peptide of SEQ ID NO: 2.

17. The peptide of claim 16 further comprising a protein tag.

18. The peptide of claim 16 wherein the serines of SEQ ID NO: 1 are phosphorylated.

19. A monoclonal or polyclonal antibody that specifically recognizes REST or any fragment thereof when the serines of SEQ ID NO: 1 are phosphorylated but does not recognize REST or any fragment thereof when the serines of SEQ ID NO: 1 are not phosphorylated.

20. The antibody of claim 20 wherein the antibody is generated through immunization with a peptide of SEQ ID NO: 4 wherein the serines of the peptide are phosphorylated.


SEQUENCE LISTING (4 sequences)

SEQ ID NO: 1 - protein, 101 aa, Artificial Sequence
  Synthetic Peptide
ISKKPPLRKDKKEKSNMQSERARKEQVLIEVGLVPVKDSWLLKESVSTEDLSPPSPPLPKENLREEASGDQKLLNTGEGNKEAPLQKVGAEEADESLPGLA

SEQ ID NO: 2 - protein, 101 aa, Artificial Sequence
  Synthetic peptide
ISKKPPLRKDKKEKSNMQSERARKEQVLIEVGLVPVKDSWLLKESVSTEDLAPPAPPLPKENLREEASGDQKLLNTGEGNKEAPLQKVGAEEADESLPGLA

SEQ ID NO: 3 - protein, 13 aa, Artificial Sequence
  Synthetic Peptide
TEDLSPPSPPLPK

SEQ ID NO: 4 - protein, 261 aa, Homo sapiens
MDSSAVITQISKEEARGPLRGKGDQKSAASQKPRSRGILHSLFCCVCRDDGEALPAHSGAPLLVEENGAIPKQTPVQYLLPEAKAQDSDKICVVIDLDETLVHSSFKPVNNADFIIPVEIDGVVHQVYVLKRPHVDEFLQRMGELFECVLFTASLAKYADPVADLLDKWGAFRARLFRESCVFHRGNYVKDLSRLGRDLRRVLILDNSPASYVFHPDNAVPVASWFDNMSDTELHDLLPFFEQLSRVDDVYSVLRQPRPGS